(19) United States
(12) Patent Application Publication
(10) Pub. No.: US 2020/0237937 A1
(43) Pub. Date: Jul. 30, 2020

(54) COMPOSITIONS AND METHODS FOR ASSESSING MACROPHAGE MEDIATED PATHOLOGY

(71) Applicant: Navidea Biopharmaceuticals, Inc., Dublin, OH (US)

(72) Inventors: David A. Ralph, Columbus, OH (US); Michael Rosol, Dublin, OH (US); Ahmad Ismail, Dublin, OH (US); Allison Kissling, Columbus, OH (US); Frederick O. Cope, Dublin, OH (US); Bonnie Chandler Abbruzzese, Dublin, OH (US)

(21) Appl. No.: 16/773,512

(22) Filed: Jan. 27, 2020

Related U.S. Application Data
(60) Provisional application No. 62/796,879, filed on Jan. 25, 2019.

(51) Int. Cl. A61K 51/06

(52) U.S. Cl. CPC A61K 51/065

(57) ABSTRACT

Compositions and methods are disclosed for assessing macrophage involvement in the inflammation of one or more joints of a subject. In certain aspects, the disclosed method includes the steps of administering to the subject a composition comprising a mannosylated dextran construct with an imaging moiety conjugated thereto; acquiring one or more planar images of a first joint of the subject; defining a region of interest (ROI) comprising the first joint; defining a joint specific reference region (RR); determining a MARTAD value of the joint by assessing the ratio of average pixel intensity of the ROI to the average pixel intensity of the RR; and comparing the MARTAD of the first joint to a normal MARTAD value for a corresponding joint, and wherein macrophage involvement is indicated by a joint specific MARTAD value that exceeds the normal MARTAD value by a predetermined threshold.

### CROSS-REFERENCE TO RELATED APPLICATION(S)

This Application claims the benefit of U.S. Provisional Application No. 62/796,879 filed on Jan. 25, 2019, which is incorporated herein by reference in its entirety

### BACKGROUND

Macrophages are immune cells that are centrally involved in many inflammatory conditions. In some circumstances, the macrophage involvement in inflammation becomes maladaptive and self-propagating, leading to chronic pathologies such as, but not limited to, cancer, atherosclerosis, and rheumatoid arthritis (RA). The mannose receptor (CD206) is highly upregulated on phenotypically activated macrophages that contribute mechanistically to the underlying pathobiology of these diseases. There is a need in the art in for a non-invasive, quantitative imaging modality for assessing macrophage involvement in inflammation that is not constrained by the subjective limitations of clinical observations.

### BRIEF SUMMARY OF THE INVENTION

Disclosed herein are compositions and methods of assessing macrophage involvement in the inflammation of one or more joints of a subject. In certain aspects, the disclosed method includes the steps of administering to the subject a composition comprising a mannosylated dextran construct with an imaging moiety conjugated thereto; acquiring one or more planar images of a first joint of the subject; defining a region of interest (ROI) comprising the first joint; defining a joint specific reference region (RR) of substantially similar size as the ROI comprising an area approximately adjacent to the ROI; determining a MARTAD value of the joint by assessing the ratio of average pixel intensity of the ROI to the average pixel intensity of the RR; and comparing the MARTAD of the first joint to a normal MARTAD value for a corresponding joint, wherein the normal MARTAD value is derived from averaging the MARTAD values for the corresponding joint from a plurality of healthy subjects and wherein macrophage involvement is indicated by a joint specific MARTAD value that exceeds the normal MARTAD value by a predetermined threshold. In some embodiments, the conjugated imaging moiety is radioactive. In some other embodiments, the imaging moiety is fluorescent. In certain aspects, the method further involves acquiring one or more planar images of one or more additional joints of the subject and repeating the foregoing steps with respect to the one or more additional joints.

According to certain embodiments, the method further includes determining a global MARTAD value of the subject. In these embodiments, the global MARTAD value is determined by quantifying the sum of difference of each of the joints of the subject that exceeds the predetermined threshold and the corresponding joint normal MARTAD value.

According to further aspects, the one or more planar image comprise at least two images and the at least two images comprise an anterior image and posterior image of the joint. In exemplary aspects of these embodiments, the subject's MARTAD value is determined by averaging the MARTAD values determined from the anterior and posterior images. In further exemplary aspects, for each joint with a MARTAD value that is within 20% of the predetermined threshold using a single planar image, the MARTAD value is recalculated using an anterior and posterior planar image.

According to certain further aspects, the joint specific RR is located within 3 ROI diameters of the ROI. In yet further aspects, the joint specific RR is located within 2 ROI diameters of the ROI.

In yet further aspects, the predetermined threshold subject joint MARTAD value is greater than or equal to two standard deviations of the average MARTAD value corresponding joint from the plurality of healthy subjects. In still further aspects, the predetermined threshold subject joint MARTAD value greater above the 95% confidence interval of the average MARTAD value corresponding joint from the plurality of healthy subjects.

In certain aspects, the mannosylated dextran construct is Tc99m-tilmanocept. In exemplary embodiments, the quantity of Tc99m-tilmanocept administered is between about 50 μg and about 400 μg. In further aspects, the time period between Tc 99m tilmanocept administration and obtaining the image of the subject is from about 15 minutes to about 6 hours.

Further disclosed herein is method of quantifying macrophage mediated joint inflammation in a subject diagnosed with rheumatoid arthritis (RA) comprising administering to the subject a composition comprising a mannosylated dextran construct and a imaging moiety conjugated thereto; selecting a plurality of joints in the subject where inflammation is suspected; acquiring one or more planar images of each of the plurality of joints; for each joint image, defining a region of interest (ROI) comprising the joint; for each joint, defining a joint specific reference region (RR) of substantially similar size as the ROI and comprising an area approximately adjacent to the ROI; for each joint, determining a MARTAD value joint by assessing the ratio of average pixel intensity of the ROI to the average pixel intensity of the RR; for each joint, comparing the MARTAD of the joint to a normal MARTAD value for a corresponding joint, wherein the normal MARTAD value is derived from averaging the MARTAD values for the corresponding joint from a plurality of healthy subjects: and wherein macrophage involvement is indicated by a joint specific MARTAD value that exceeds the normal MARTAD value by a predetermined threshold; and determining a global MARTAD value for the subject by determining the sum of difference of each of the joints of the subject that exceeds the predetermined threshold and the corresponding joint normal MARTAD value.

Further disclosed herein is a method of managing the treatment of a subject diagnosed with RA comprising the steps of administering to the subject a composition comprising a mannosylated dextran construct and an imaging moiety conjugated thereto; selecting a plurality of joints in the subject where inflammation is suspected; acquiring one or more planar images of each of the plurality of joints; for each joint image, defining a region of interest (ROI) comprising the joint; for each joint, defining a joint specific reference region (RR) of substantially similar size as the ROI and comprising an area approximately adjacent to the ROI; for each joint, determining a MARTAD value joint by assessing the ratio of average pixel intensity of the ROI to the average pixel intensity of the RR; for each joint, comparing the MARTAD of the joint to a normal MARTAD value for a corresponding joint, wherein the normal MARTAD value is derived from averaging the MARTAD values for the corresponding joint from a plurality of healthy subjects: and wherein macrophage involvement is indicated by a joint specific MARTAD value that exceeds the normal MARTAD value by a predetermined threshold; determining a global MARTAD value for the subject by determining the sum of difference of each of the joints of the subject that exceeds the predetermined threshold and the corresponding joint normal MARTAD value; and administering a course of treatment to the subject. In certain aspects, following the course of treatment, the foregoing step are repeated and the change in global MARTAD score is evaluated. In exemplary implementations, a decrease in global MARTAD value is indicative of efficacy of the course of treatment.

### BRIEF DESCRIPTION OF THE FIGURES

FIG. 1 shows exemplary images of subject hands, according to certain implementations.

FIG. 2 shows exemplary ROIs and joint specific RRs, according to certain implementations.

FIG. 3 shows exemplary MARTAD value data from RA subjects and healthy controls, according to certain embodiments.

FIG. 4 shows exemplary Global MARTAD values from 9 subjects with active RA, according to certain embodiments.

### DETAILED DESCRIPTION

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

As used herein, the term “pharmaceutically acceptable carrier” or “carrier” refers to sterile aqueous or nonaqueous solutions, colloids, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “subject” or “patient” refers to the target of administration, e.g., an animal. Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more cancer disorders prior to the administering step.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with rheumatoid arthritis” means having been subjected to a physical examination by a person of skill, for example, a physician, and found to have a condition that can be diagnosed or treated by a compound or composition that can reduce inflammation of the joints and/or the pain associated therewith.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, administration to specific organs through invasion, intramuscular administration, intratumoral administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

“Tilmanocept” refers to a non-radiolabeled precursor of the LYMPHOSEEK® diagnostic agent. Tilmanocept is a mannosylaminodextran. It has a dextran backbone to which a plurality of amino-terminated leashes (—O(CH2)3S(CH2)2NH2) are attached to the core glucose elements. In addition, mannose moieties are conjugated to amino groups of a number of the leashes, and the chelator diethylenetriamine pentaacetic acid (DTPA) may be conjugated to the amino group of other leashes not containing the mannose. Tilmanocept generally, has a dextran backbone, in which a plurality of the glucose residues comprise an amino-terminated leash:

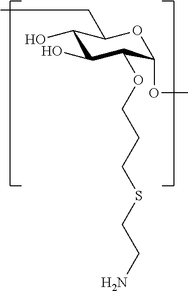

the mannose moieties are conjugated to the amino groups of the leash via an amidine linker:

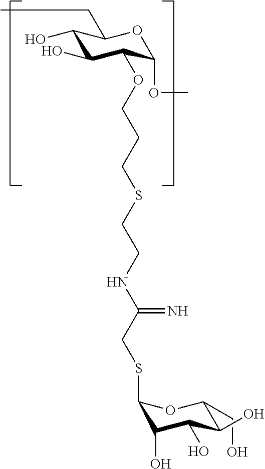

the chelator diethylenetriamine pentaacetic acid (DTPA) is conjugated to the amino groups of the leash via an amide linker:

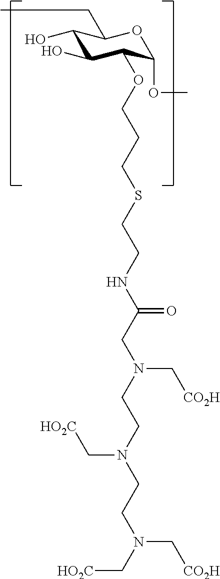

Tilmanocept has the chemical name dextran 3-[(2-aminoethyl)thio]propyl 17-carboxy-10,13,16-tris(carboxymethyl)-8-oxo-4-thia-7,10,13,16-tetraazaheptadec-1-yl 3-[[2-[[1-imino-2-(D-mannopyranosylthio)ethyl]amino]ethyl]thio]propyl ether complexes, and tilmanocept Tc99m has the following molecular formula:

[C6H10O5]n.(C19H28N4O9S99mTc)b.(C13H24N2O5S2)c.(C5H11NS)a and contains 3-8 conjugated DTPA molecules (b); 12-20 conjugated mannose molecules (c); and 0-17 amine side chains (a) remaining free. Tilmanocept has the following general structure:

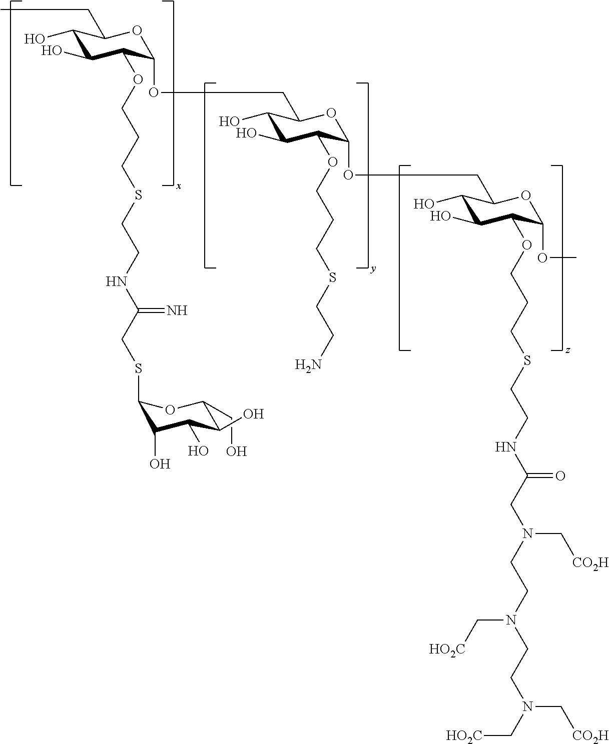

Certain of the glucose moieties may have no attached amino-terminated leash.

Disclosed herein is a method to quantify the amount of macrophage involved disease activity in a particular anatomical region of interest and to quantitatively determine how macrophage involvement changes over time and in response to therapies. Certain embodiments described herein disclose the use of an imaging agent, Tc99m-tilmanocept, to quantify macrophage involvement in inflammation at particular sites of interest. Tilmanocept is a mannosylated dextran synthetic molecular construct with high affinity and specificity for the mannose receptor, CD206. Certain embodiments disclose quantifying macrophage involvement in inflamed joints of the hands and wrists in patients with RA. However, the utility of the disclosed methods is not limited to these examples. The present invention describes a method to quantify macrophage involvement at sites of inflammation using any mannosylated dextran construct, which can be detected through conjugation or association with many various radioisotopic or fluorescent moieties. The instantly disclosed methods have utility for evaluating macrophage involvement in many disease states besides RA, including cancer and atherosclerosis.

In certain embodiments, the disclosed methods measure the global macrophage involved disease activity across all, or substantially all, relevant joints in an RA patient. In a typical RA patient, a significant portion of evaluated joints, but rarely all evaluated joints, are involved in disease activity (i.e. RA caused inflammation). Over time, the disease activity in any individual joint may increase or decrease, and joints that were previously not involved may develop RA inflammation. In the present invention, a method to assess and quantify the amount of macrophage mediated RA disease involvement across all joints is disclosed. This method has utility for monitoring disease activity in an RA patient over time and for providing a quantitative measurement of responses to newly initiated RA therapies. In addition, a quantitative global measurement of the degree of macrophage involvement across all evaluated joints may facilitate choosing which of many RA therapies an individual RA patient is most likely to respond.

Disclosed herein are compositions and methods of assessing macrophage involvement in the inflammation of one or more joints of a subject. In certain aspects, the disclosed method includes the steps of administering to the subject a composition comprising a mannosylated dextran construct and radioactive imaging moiety conjugated thereto; acquiring one or more planar images of a first joint of the subject; defining a region of interest (ROI) comprising the first joint; defining a joint specific reference region (RR) of substantially similar size as the ROI comprising an area approximately adjacent to the ROI; determining a MARTAD value of the joint by assessing the ratio of average pixel intensity of the ROI to the average pixel intensity of the RR; and comparing the MARTAD of the first joint to a normal MARTAD value for a corresponding joint, wherein the normal MARTAD value is derived from averaging the MARTAD values for the corresponding joint from a plurality of healthy subjects and wherein macrophage involvement is indicated by a joint specific MARTAD value that exceeds the normal MARTAD value by a predetermined threshold. In certain aspects, the method further involves acquiring one or more planar images of one or more additional joints of the subject and repeating the foregoing steps with respect to the one or more additional joints. mannosylated dextran construct

In certain aspects, compounds disclosed herein employ a carrier construct comprising a polymeric (e.g. carbohydrate) backbone having conjugated thereto mannose-binding C-lectin type receptor targeting moieties (e.g. mannose) to deliver one or more active therapeutic agent. Examples of such constructs include mannosylamino dextrans (MAD), which comprise a dextran backbone having mannose molecules conjugated to glucose residues of the backbone and having an active pharmaceutical ingredient conjugated to glucose residues of the backbone. Tilmanocept is a specific example of an MAD. A tilmanocept derivative that is tilmanocept without DTPA conjugated thereto is a further example of an MAD.

In certain implementations, the disclosure provides a compound comprising a dextran-based moiety or backbone having one or more mannose-binding C-type lectin receptor targeting moieties and one or more therapeutic agents attached thereto. The dextran-based moiety generally comprises a dextran backbone similar to that described in U.S. Pat. No. 6,409,990 (the '990 patent), which is incorporated herein by reference. Thus, the backbone comprises a plurality of glucose moieties (i.e., residues) primarily linked by α-1,6 glycosidic bonds. Other linkages such as α-1,4 and/or α-1,3 bonds may also be present. In some embodiments, not every backbone moiety is substituted. In some embodiments, mannose-binding C-type lectin receptor targeting moieties are attached to between about 10% and about 50% of the glucose residues of the dextran backbone, or between about 20% and about 45% of the glucose residues, or between about 25% and about 40% of the glucose residues.

According to further aspects, the mannose-binding C-type lectin receptor targeting moiety is selected from, but not limited to, mannose, fucose, and n-acetylglucosamine. In some embodiments, the targeting moieties are attached to between about 10% and about 50% of the glucose residues of the dextran backbone, or between about 20% and about 45% of the glucose residues, or between about 25% and about 40% of the glucose residues. MWs referenced herein, as well as the number and degree of conjugation of receptor substrates, leashes, and diagnostic/therapeutic moieties attached to the dextran backbone refer to average amounts for a given quantity of carrier molecules, since the synthesis techniques will result in some variability.

According to certain embodiments, the one or more mannose-binding C-type lectin receptor targeting moieties and one or more detectable agents (e.g. a radiolabeled imaging moiety) are attached to the dextran-based moiety by way of a linker. The linker may be attached at from about 50% to about 100% of the backbone moieties or about 70% to about 90%. The linkers may be the same or different. In some embodiments, the linker is an amino-terminated linker. In some embodiments, the linkers may comprise —O(CH2)3S(CH2)2NH—. In some embodiments, the linker may be a chain of from 1 to 20 member atoms selected from carbon, oxygen, sulfur, nitrogen and phosphorus. The linker may be a straight chain or branched. The linker may also be substituted with one or more substituents including, but not limited to, halo groups, perfluoroalkyl groups, perfluoroalkoxy groups, alkyl groups, such C1-4 alkyl, alkenyl groups, such as C1-4 alkenyl, alkynyl groups, such as C1-4 alkynyl, hydroxy groups, oxo groups, mercapto groups, alkylthio groups, alkoxy groups, nitro groups, azidealkyl groups, aryl or heteroaryl groups, aryloxy or heteroaryloxy groups, aralkyl or heteroaralkyl groups, aralkoxy or heteroaralkoxy groups, HO—(C═O)— groups, heterocylic groups, cycloalkyl groups, amino groups, alkyl- and dialkylamino groups, carbamoyl groups, alkylcarbonyl groups, alkylcarbonyloxy groups, alkoxycarbonyl groups, alkylaminocarbonyl groups, dialkylamino carbonyl groups, arylcarbonyl groups, aryloxycarbonyl groups, alkylsulfonyl groups, arylsulfonyl groups, —NH—NH2; ═N—H; ═N-alkyl; —SH; —S-alkyl; —NH—C(O)—; —NH—C(═N)— and the like. As would be apparent to one skilled in the art, other suitable linkers are possible.

The disclosed compounds can include an imaging moiety or detectable label. As used herein, the term “imaging moiety” means an atom, isotope, or chemical structure which is: (1) capable of attachment to the carrier molecule; (2) non-toxic to humans or other mammalian subjects; and (3) provides a directly or indirectly detectable signal, particularly a signal which not only can be measured but whose intensity is related (e.g., proportional) to the amount of the imaging moiety. The signal may be detected by any suitable means, including spectroscopic, electrical, optical, magnetic, auditory, radio signal, or palpation detection means.

Imaging moieties include, but are not limited to, radioactive isotopes (radioisotopes), fluorescent molecules (a.k.a. fluorochromes and fluorophores), chemiluminescent reagents (e.g., luminol), bioluminescent reagents (e.g., luciferin and green fluorescent protein (GFP)), and metals (e.g., gold nanoparticles). Suitable imaging moieties can be selected based on the choice of imaging method. For example, the detection label can be a near infrared fluorescent dye for optical imaging, a Gadolinium chelate for MRI imaging, a radionuclide for PET or SPECT imaging, or a gold nanoparticle for CT imaging.

Imaging moieties can be selected from, for example, a radionuclide, a radiological contrast agent, a paramagnetic ion, a metal, a fluorescent label, a chemiluminescent label, an ultrasound contrast agent, a photoactive agent, or a combination thereof. Non-limiting examples of imaging moieties include a radionuclide such ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{(4m}Tc, ⁹⁴Tc, ^{99m}Tc, ¹²⁰I, ¹²³I, ¹²⁴I, ¹²⁵I, ¹³¹I, ¹⁵⁴⁻¹⁵⁸G, ³²P, ¹¹C, ¹³N, ¹⁵O, ¹⁸⁹Re, ¹⁸⁸Re, ⁵¹Mn, ^{52m}Mn, ⁵⁵Co, ⁷²As, ⁷⁶Br, ^{82m}Rb, ⁸³Sr, ^{117m}Sn or other gamma-, beta-, or positron-emitters. Gamma radiation from radioisotopes can be detected using a gamma particle detection device. In some embodiments, the gamma particle detection device is a Gamma Finder® device (SenoRx, Irvine, Calif.). In some embodiments, the gamma particle detection device is a Neoprobe® GDS gamma detection system (Dublin, Ohio).

Paramagnetic ions of use may include chromium (III), manganese (II), iron (H), iron (II), cobalt (II), nickel (II), copper (II), neodymium (III), samarium (III), ytterbium (III), gadolinium (III), vanadium (II), terbium (III), dysprosium (III), holmium (III) or erbium (III). Metal contrast agents may include lanthanum (III), gold (III), lead (II) or bismuth (III). Ultrasound contrast agents may comprise liposomes, such as gas-filled liposomes.

Other suitable labels include, for example, fluorescent labels (such as GFP and its analogs, fluorescein, isothiocyanate, rhodamine, phycoerythrin, phycocyanin, allophycocyanin, o-phthaldehyde, and fluorescamine and fluorescent metals such as Eu or others metals from the lanthanide series), near IR dyes, quantum dots, phosphorescent labels, chemiluminescent labels or bioluminescent labels (such as luminal, isoluminol, theromatic acridinium ester, imidazole, acridinium salts, oxalate ester, or dioxetane).

In certain aspects, the mannosylated dextran construct is Tc99m-tilmanocept. In exemplary implementations of these embodiments, the intended route of administration for Tc 99m tilmanocept is intravenous (IV). In some embodiments the site of IV placement is the left or right antecubital vein. In some embodiments, the IV placement site is between the elbow and wrist. In some embodiments, the quantity of tilmanocept administered IV is between about 50 μg and about 400 μg. In some embodiments, the Tc 99m radiolabeling ranges from about 1 mCi to about 10 mCi. In some embodiments, the Tc 99m tilmanocept is administered IV in one dose. In some embodiments, the Tc 99m tilmanocept is administered IV in more than one dose. In some embodiments, following administration of the Tc 99m tilmanocept, sterile saline is administered. In further aspects, the time period between Tc 99m tilmanocept administration and obtaining the image of the subject is from about 15 minutes to about 6 hours.

### Acquiring One or More Planar Images

According to certain embodiments, one or more planar images are acquired after a defined time interval following administration of the mannosylated dextran compound. In some embodiments, the time between administration and acquisition of images is between about 15 minutes to about 6 hours. In some embodiments, the time between administration and acquisition of images is between about 15 minutes to about 3 hours. In some embodiments, the time between administration and acquisition of images is between about 1 hour to about 3 hours or more. In some embodiments, the time between administration and acquisition of images is between about 4 hours to about 6 hours or more.

In some embodiments, the camera used to acquire planar images for analysis is a dual-headed SPECT or SPECT/CT camera equipped with a low-energy, high-resolution collimator with a 15% window (20% can be used if 15% setting not available), and in certain implementations where Tc99m-tilmanocept is administered, centered over a 140 keV peak. In some embodiments, a target of 5-7 million counts is obtained using state-of-the-art 2-headed cameras (nominal 20″×15″ FOV). According to further implementations, a single headed camera is used for image acquisition. According to certain alternative embodiments, image acquisition period is based on time rather than counts. In exemplary implementations, image acquisition occurs during a window of, for example, about 5 to about 20 minutes. Shorter or longer time periods are possible. In certain embodiments, whole body scans are performed. In further embodiments, only the hands, only the feet, or only the hands and feet are scanned. In the foregoing embodiments, where only the hands and/or feet are scanned, image acquisition time periods are generally a shorter duration than when the whole body is scanned.

### Defining ROI

According to certain embodiments, following image acquisition, one or more regions of interest (ROI) are defined. In certain aspects, the ROI is subset of the pixels of the full image that contains the anatomical region to be assessed (e.g., a joint). In certain embodiments, an ROI is defined by a health care provider. In alternative embodiments, the ROI is defined by, or with the assistance of a computer implemented algorithm. In exemplary aspects of these embodiments, the algorithm my employ machine learning to improve accuracy of ROI selection.

In some embodiments, intermeans thresholding is used for selecting the ROI. In some embodiments, the ROI is selected manually by drawing an area. In some embodiments, for example in RA, the ROI is manually drawn around a joint. In some embodiments, manual ROIs are drawn tightly around the joint to minimize potential signal dilution from extraneous soft tissue. From these ROIs, the average and/or maximum pixel intensity is obtained, which represents the quantification of disease-specific activated macrophage activity within the ROI.

Several commercial and open-source packages are available for the quantitation of medical images. For example, ImageJ is an open-architecture, Java-based program developed by the National Institutes of Health (NIH) compatible with Macintosh, Linux, and Windows operating systems. It is equipped with processing features including the calculation of area and pixel value statistics from defined regions, image windowing (i.e., adjust brightness/contrast) for greater visualization without modifying true quantitative data, and the ability to cut, copy, or paste images or selections. ImageJ can open and save a variety of image file extensions including DICOM (Digital Imaging and Communications in Medicine) images.

### Defining RR

In certain aspects, the disclosed method comprises defining a refence region. In exemplary embodiments, reference region is a joint-specific reference region. That is, the reference region selected is matched specifically to the ROI in terms of anatomical proximity and/or size. According to certain implementations, the joint-specific RR is adjacent to the ROI. In further implementations, the RR is approximately adjacent to the ROI. In exemplary implementations of these embodiments, the RR is about 3 ROI diameters or less from the closest edge of the ROI. In further implementations, the RR is about 2 ROI diameters or less from the closest edge of the ROI.

According to certain embodiments, the joint-specific reference region is the same size, or substantially the same size, as the ROI.

According to certain alternative embodiments, and specifically, certain embodiments where the ROI is one or more of the joints of the hands or feet, the RR is defined as an area containing multiple joints of the hands or feet, less the pixel intensity value of the ROIs within the RR. According to certain implementations of these embodiments, the RR is defined entire hand, minus the pixel intensity of the MCPs and PIPs. In further embodiments, the RR is defined as an area containing a subset of MCPs and/or PIPs, minus the pixel intensities of the MCPs and PIPs contained with the RR area. In certain implementations, these larger MCP specific reference region may have less observational variation relative to the individual MCPs than smaller joint specific reference regions. Similar joint type specific reference regions could be drawn for the PIP and wrist joint classes. Reducing the observational variation in the reference region may reduce observational variation in joint specific MARTAD values.

### Determining MARTAD Value

In certain aspects, the pixel intensities of the ROI and RR are used to derive normalized region-specific mannose receptor targeted localization determination (MARTAD) value. The MARTAD value is quantitative index of the amount of imaging agent localization that can be attributable to disease activity in planar images. Defined broadly, the MARTAD value is determined by assessing the ratio of average pixel intensity of the ROI to the average pixel intensity of the RR. In certain alternative embodiments, the MARTAD value is determined by assessing the ratio of maximum pixel intensity of the ROI to the maximum pixel intensity of the RR.

Pixel intensity determinations can be made through many commercial and open-source packages available for the quantitation of medical images known in the art. For example, the RadiAnt DICOM viewer software (v. 5.0.2). Alternatively, the ImageJ program can be used to quantify ROIs and RRs and summarizes area and intensity values as pixel statistics. These pixel statistics include pixel area, mean intensity, minimum intensity, maximum intensity, and median intensity of the ROI and/or RR.

### Determining Macrophage Involvement

Following MARTAD value determination for a joint or plurality of joints, the MARTAD value can be used to determine macrophage involvement by comparing the MARTAD of the first joint to a normal MARTAD value for a corresponding joint (e.g., RtPIP2 RA vs RtPIP2 healthy). In certain implementations, the normal MARTAD value is determined by aggregating the MARTAD values for each joint from a pool of healthy subjects (e.g., not suffering from RA). In exemplary implementations, the method for defining the joint specific RR in the pool of healthy subjects will be the same as that used for the patient population. Macrophage involvement in a subject is indicated by a joint specific MARTAD value that exceeds the normal MARTAD value by a predetermined threshold. In certain implementations, the predetermined threshold is exceeded when the subject joint MARTAD value is greater than or equal to two standard deviations of the average MARTAD value of the corresponding joint from the plurality of healthy subjects. In certain alternative implementations, the predetermined threshold subject joint MARTAD value greater above the 95% confidence interval of the average MARTAD value corresponding joint from the plurality of healthy subjects.

According to certain embodiments, the method further includes determining a global MARTAD value of the subject. In these embodiments, the global MARTAD value is determined by quantifying the sum of difference of each of the joints of the subject that exceeds the predetermined threshold and the corresponding joint normal MARTAD value. Global MARTAD values provide utility for those managing patients with RA in at least the following respects. First, RA patients with low macrophage involvement in their RA inflammation may be less likely to respond to certain specific RA therapies. Global MARTAD values facilitate identifying to which RA patients these therapies are most likely to be effective. Second, macrophage numbers decline rapidly and prior to reductions in clinical symptoms in those RA patients who are responding to newly initiated therapies, allowing changes in MARTAD values to provide an early quantitative indication that a therapy will be effective. Third, as Global MARTAD values are quantitative, they provide an objective means to monitor RA patients over time for the extent of their macrophage involved RA inflammation.

According to further aspects, planar images comprising at least an anterior image and a posterior image of a joint and its joint specific reference region are evaluated. In exemplary aspects of these embodiments, the subject's MARTAD value is determined by averaging the MARTAD values determined from the anterior and posterior images. In further exemplary aspects, MARTAD values are calculated for all evaluated joints for both the anterior and posterior views with the higher MARTAD value accepted for further analyses. In further exemplary aspects, for each joint with a MARTAD value that is within 20% of the predetermined threshold using a single planar image, the MARTAD value is recalculated using an anterior and posterior planar image.

Further disclosed herein is method of quantifying macrophage mediated joint inflammation in a subject diagnosed with rheumatoid arthritis (RA) comprising administering to the subject a composition comprising a mannosylated dextran construct and an imaging moiety conjugated thereto; selecting a plurality of joints in the subject where inflammation is suspected; acquiring one or more planar images of each of the plurality of joints; for each joint image, defining a region of interest (ROI) comprising the joint; for each joint, defining a joint specific reference region (RR) of substantially similar size as the ROI and comprising an area approximately adjacent to the ROI; for each joint, determining a MARTAD value joint by assessing the ratio of average pixel intensity of the ROI to the average pixel intensity of the RR; for each joint, comparing the MARTAD of the joint to a normal MARTAD value for a corresponding joint, wherein the normal MARTAD value is derived from averaging the MARTAD values for the corresponding joint from a plurality of healthy subjects: and wherein macrophage involvement is indicated by a joint specific MARTAD value that exceeds the normal MARTAD value by a predetermined threshold; and determining a global MARTAD value for the subject by determining the sum of difference of each of the joints of the subject that exceeds the predetermined threshold and the corresponding joint normal MARTAD value.

Further disclosed herein is a method of managing the treatment of a subject diagnosed with RA comprising the steps of administering to the subject a composition comprising a mannosylated dextran construct and an imaging moiety conjugated thereto; selecting a plurality of joints in the subject where inflammation is suspected; acquiring one or more planar images of each of the plurality of joints; for each joint image, defining a region of interest (ROI) comprising the joint; for each joint, defining a joint specific reference region (RR) of substantially similar size as the ROI and comprising an area approximately adjacent to the ROI; for each joint, determining a MARTAD value joint by assessing the ratio of average pixel intensity of the ROI to the average pixel intensity of the RR; for each joint, comparing the MARTAD of the joint to a normal MARTAD value for a corresponding joint, wherein the normal MARTAD value is derived from averaging the MARTAD values for the corresponding joint from a plurality of healthy subjects: and wherein macrophage involvement is indicated by a joint specific MARTAD value that exceeds the normal MARTAD value by a predetermined threshold; determining a global MARTAD value for the subject by determining the sum of difference of each of the joints of the subject that exceeds the predetermined threshold and the corresponding joint normal MARTAD value; administering a course of treatment to the subject. Following the course or treatment, global MARTAD values are reassessed through the methods provided herein. In certain implementations, a decrease in global MARTAD value is indicative of efficacy of the course of treatment.

A “course of treatment” can be any treatment known in the art to be effective for the treatment of RA. For example, the conventionally well-known therapeutic drugs include biological preparations, non-steroidal anti-inflammatory drugs (anti-inflammatory analgesics), steroidal drugs and immunosuppressants. The biological preparations include chimeric anti-TNF-alpha antibody preparations, soluble TNF receptors, fully human anti-TNF-alpha antibody preparations and anti-IL-6 receptor antibody preparations. The non-steroidal anti-inflammatory drugs include prostaglandin synthesis inhibitors. More specifically, the therapeutic drugs for rheumatoid arthritis according to the present invention include, but are not limited to, methotrexate (MTX), infliximab (IFX), etanercept (ETN), tocilizumab (TCZ), adalimumab (ADA) and abatacept (ABT).

In certain embodiments, treatment induced changes in the global MARTAD score are evaluated in conjunction with changes in the subject's clinical presentation. In certain implementations, the Disease Activity Score (DAS) (Fransen & van Riel Clin Exp Rheumatol 23:S93-S99 2005) is used. The DAS is calculated by a medical practitioner based on various validated measures of disease activity, including physical symptoms of RA. A reduction in DAS reflects a reduction in disease severityDAS28 is the Disease Activity Score in which 28 joints in the body are assessed to determine the number of tender joints and the number of swollen joints (Prevoo et al. Arthritis Rheum 38:44-48 1995). The American College of Rheumatology (ACR) proposed a set of criteria for classifying RA. The commonly used criteria are the ACR 1987 revised criteria (Arnett et al. Arthritis Rheum. 31:315-324 1988). Diagnosis of RA according to the ACR criteria requires a patient to satisfy a minimum number of listed criteria, such as tender or swollen joint counts, stiffness, pain, radiographic indications and measurement of serum rheumatoid factor. An individual, patient reported measure of disability in RA patients is the Health Assessment Questionnaire Disability Index (HAQ-DI). HAQ-DI scores represent physical function in terms of the patient's reported ability to perform everyday tasks, including the level of difficulty they experience in carrying out the activity. By recording patients' ability to perform everyday activities, the HAQ-DI score can be used as one measure of their quality of life. In some embodiments, the pixel intensity values are maximum pixel intensity values. In some embodiments, the pixel intensity values are average pixel intensity values.

### EXAMPLES

The following examples are put forth so as to provide those of ordinary skill in the art with a complete disclosure and description of certain examples of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

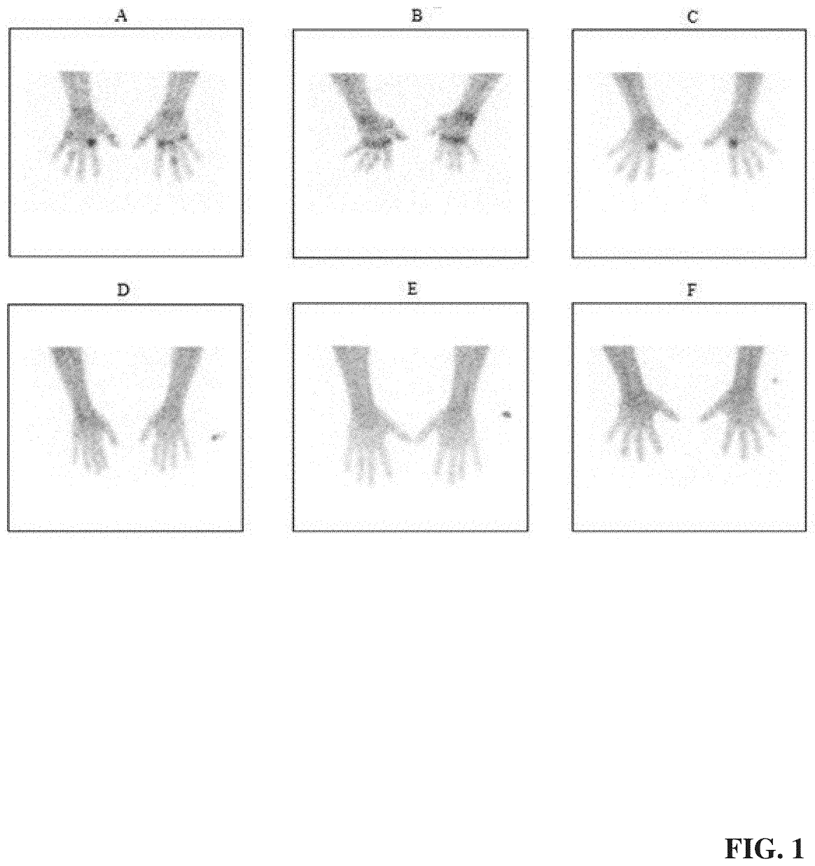

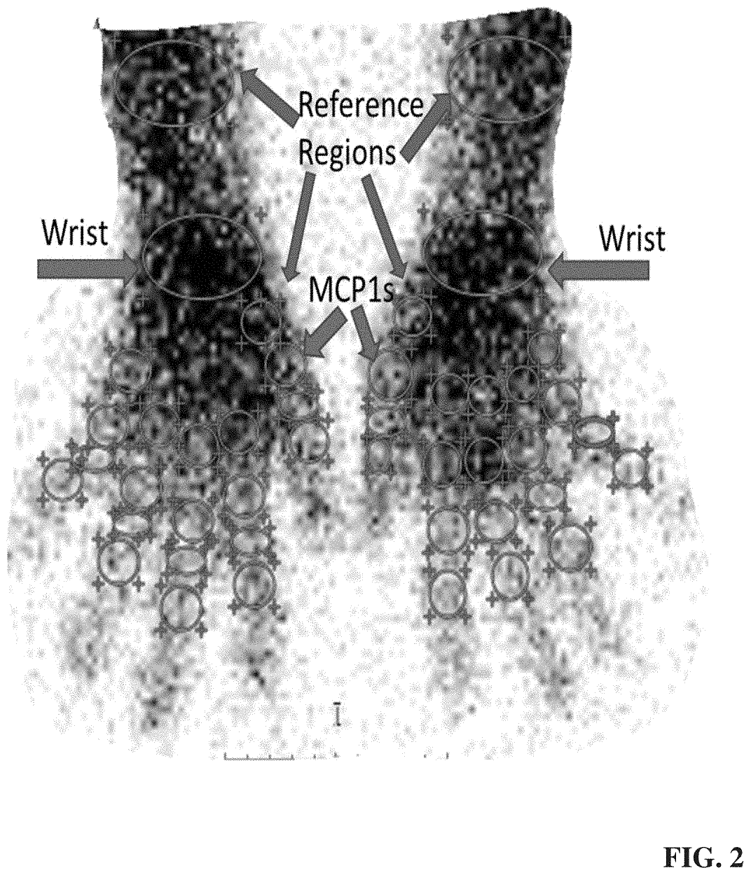

All results described in the example were obtained from images acquired of subjects that had been injected intravenously (IV) with Lymphoseek® that had been labeled with 10 mCi of 99m-technetium. Lymphoseek is a commercially available imaging agent drug product in which tilmanocept is the active ingredient. The mass dose of tilmanocept injected into the subjects varied from 125 μg to 400 μg. Separate studies (not shown) demonstrated that varying the mass dose over this range at did not impact image quality. All images evaluated in the examples were acquired 45-75 minutes after IV administration of the Tc99m-tilmanocept; however, repeat images acquired 180-240 minutes after injection in the same subjects produced statistically similar MARTAD values. Different gamma cameras located at different imaging facilities have been used to image RA subjects and healthy control subjects. The images shown in FIGS. 1 and 2 were acquired using a Siemens Symbia Intevo camera. Image acquisition times varied between studies from 5-10 minutes. MARTAD values were not sensitive to image acquisition times that varied over this range. Shorter or longer image acquisition times may have also been acceptable.

As best shown in FIG. 1, RA subjects (top row: A, B, C) or healthy control subjects without RA (bottom row: D, E, F) injected intravenously (IV) with either 200 (A) or 400 (all others) micrograms of tilmanocept labeled with 10 mCi of 99mtechnetium (Tc99m). Planar gamma images were acquired 60 minutes after injection.

FIG. 2 shows exemplary ROIs (joints) and joint specific RR drawn on a planar image of the left and right hands of a subject with RA. Arrows indicate the RR and RIOs for the wrists and MCP1s.

Table 1 shows the analysis of data Mean MARTAD values and their S.D. calculated from 5 healthy subjects each injected intravenously with tilmanocept labeled with 10 mCi of 99mTc. Cutoff values for determination of macrophage involvement in RA inflammation set at MARTAD values greater than 2 S.D. above the joint specific mean.

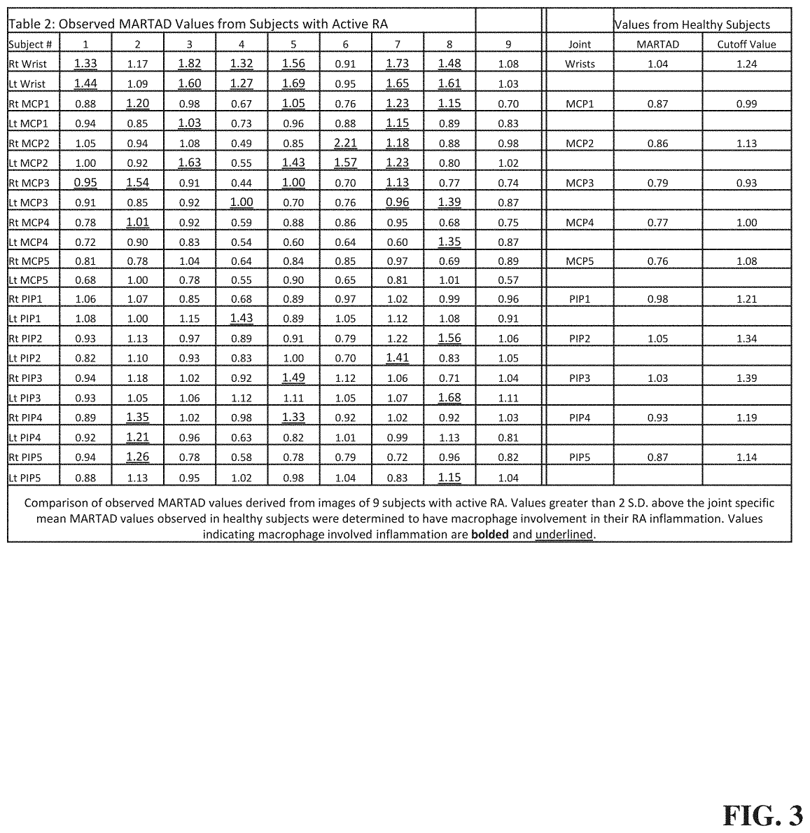

Table 2, as shown in FIG. 3, shows the comparison of observed MARTAD values derived from images of 9 subjects with active RA. Values greater than 2 S.D. above the joint specific mean MARTAD values observed in healthy subjects were determined to have macrophage involved inflammation are shaded.

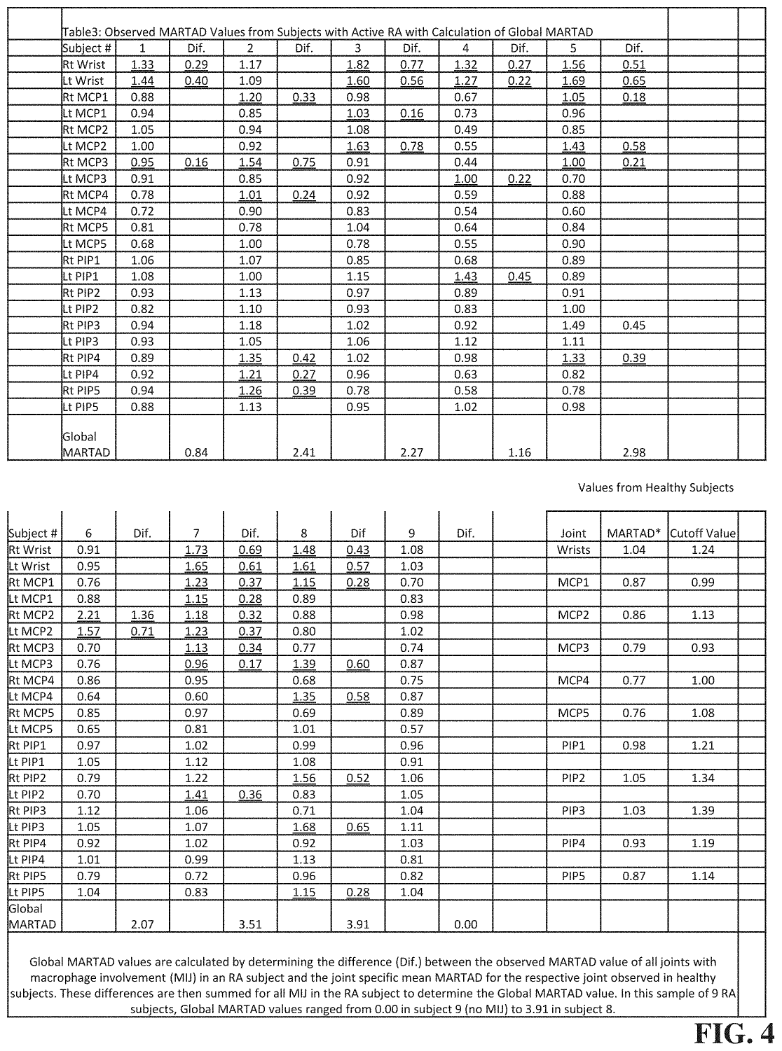

Table 3, as shown in FIG. 4, shows Global MARTAD values calculated by determining the difference (Dif.) between the observed MARTAD value of all joints with macrophage involvement (MJI) in an RA subject and the joint specific mean MARTAD for the respective joint observed in healthy subjects. These differences were then summed for all MJI in the RA subject to determine the Global MARTAD value. In this sample of 9 RA subject, Global MARTAD values range from 0.00 in subject 9 (no MJI) to 3.91 in subject 8.

TABLE 1
Mean Joint Specific MARTAD values, the Standard Deviations of
the Means, and Cutoff values Established from Healthy Subject

| Joint | Mean Joint Specific | | Cutoff |
|---|---|---|---|
| Type | MARTAD | S.D. | Value |
| Wrists | 1.04 | 0.10 | 1.24 |
| MCP1 | 0.87 | 0.06 | 0.99 |
| MCP2 | 0.86 | 0.13 | 1.13 |
| MCP3 | 0.79 | 0.07 | 0.93 |
| MCP4 | 0.77 | 0.12 | 1.00 |
| MCP5 | 0.76 | 0.16 | 1.08 |
| PIP1 | 0.98 | 0.12 | 1.21 |
| PIP2 | 1.05 | 0.15 | 1.34 |
| PIP3 | 1.03 | 0.18 | 1.39 |
| PIP4 | 0.93 | 0.13 | 1.19 |
| PIP5 | 0.87 | 0.14 | 1.14 |

Mean MARTAD values and their S.D. calculated from 5 healthy subjects each injected intravenously with tilmanocept labeled with 10 mCi of 99 mTc. Cutoff values for determination of macrophage involvement in RA inflammation set at MARTAD values greater than 2 S.D. above the joint Specific mean.

What is claimed is:

1. A method of assessing macrophage involvement in the inflammation of one or more joints of a subject comprising:
a. administering to the subject a composition comprising a mannosylated dextran construct imaging moiety conjugated thereto;
b. acquiring one or more planar images of a first joint of the subject;
c. defining a region of interest (ROI) comprising the first joint;
d. defining a joint specific reference region (RR) of substantially similar size as the ROI comprising an area approximately adjacent to the ROI;
e. determining a MARTAD value of the joint by assessing the ratio of average pixel intensity of the ROI to the average pixel intensity of the RR; and
f. comparing the MARTAD of the first joint to a normal MARTAD value for a corresponding joint, wherein the normal MARTAD value is derived from averaging the MARTAD values for the corresponding joint from a plurality of healthy subjects and wherein macrophage involvement is indicated by a joint specific MARTAD value that exceeds the normal MARTAD value by a predetermined threshold.

2. The method of claim 1, further comprising acquiring one or more planar images of one or more additional joints of the subject and repeating steps c-f with respect to the one or more additional joints.

3. The method of claim 2, further comprising determining a global MARTAD value of the subject wherein the global MARTAD value is determined by determining the sum of difference of each of the joints of the subject that exceeds the predetermined threshold and the corresponding joint normal MARTAD value.

4. The method of claim 1, wherein the one or more planar image comprise at least two images and the at least two images comprise an anterior image and posterior image of the joint and wherein the subject's MARTAD value is determined by averaging the MARTAD values determined from the anterior and posterior images.

5. The method of claim 3, wherein for each joint with a MARTAD value that is within 20% of the predetermined threshold using a single planar image, the MARTAD value is recalculated using an anterior and posterior planar image.

6. The method of claim 1, wherein the joint specific RR is located within 3 ROI diameters of the ROI.

7. The method of claim 1, wherein the imaging moiety is a radioactive imaging moiety or a fluorescent imaging moiety.

8. The method of claim 1, wherein the predetermined threshold subject joint MARTAD value is greater than or equal to two standard deviations of the average MARTAD value corresponding joint from the plurality of healthy subjects.

9. The method of claim 1, wherein the predetermined threshold subject joint MARTAD value greater above the 95% confidence interval of the average MARTAD value corresponding joint from the plurality of healthy subjects.

10. The method of claim 1, wherein the mannosylated dextran construct is Tc99m-tilmanocept.

11. The method of claim 10, wherein the quantity of Tc99m-tilmanocept administered is between about 50 μg and about 400 μg.

12. The method of claim 10, wherein the time period between Tc 99m tilmanocept administration and obtaining the image of the subject is from about 15 minutes to about 6 hours.

13. A method of quantifying macrophage mediated joint inflammation in a subject diagnosed with rheumatoid arthritis (RA) comprising:
a. administering to the subject a composition comprising a mannosylated dextran construct and an imaging moiety conjugated thereto;
b. selecting a plurality of joints in the subject where inflammation is suspected;
c. acquiring one or more planar images of each of the plurality of joints;
d. for each joint image, defining a region of interest (ROI) comprising the joint;
e. for each joint, defining a joint specific reference region (RR);
f. for each joint, determining a MARTAD value joint by assessing the ratio of average pixel intensity of the ROI to the average pixel intensity of the RR;
g. for each joint, comparing the MARTAD of the joint to a normal MARTAD value for a corresponding joint, wherein the normal MARTAD value is derived from averaging the MARTAD values for the corresponding joint from a plurality of healthy subjects: and wherein macrophage involvement is indicated by a joint specific MARTAD value that exceeds the normal MARTAD value by a predetermined threshold; and
h. determining a global MARTAD value for the subject by determining the sum of difference of each of the joints of the subject that exceeds the predetermined threshold and the corresponding joint normal MARTAD value.

14. The method of claim 13, wherein for each joint, the joint specific RR is located within 3 ROI diameters of the ROI.

15. The method of claim 14, wherein the joint specific RR is located within 2 ROI diameters of the ROI.

16. The method of claim 13, wherein the plurality of joints are joints of a hand or foot and the RR is defined as the hand or foot, less the pixel intensity of the ROIs within the RR.

17. The method of claim 13, wherein the mannosylated dextran construct is Tc99m-tilmanocept and wherein the quantity of Tc99m-tilmanocept administered is between about 50 μg and about 400 μg.

18. A method of managing treatment of a subject diagnosed with RA comprising the steps of:
a. administering to the subject a composition comprising a mannosylated dextran construct and an imaging moiety conjugated thereto;
b. selecting a plurality of joints in the subject where inflammation is suspected;
c. acquiring one or more planar images of each of the plurality of joints;
d. for each joint image, defining a region of interest (ROI) comprising the joint;
e. for each joint, defining a joint specific reference region (RR) of substantially similar size as the ROI and comprising an area approximately adjacent to the ROI;
f. for each joint, determining a MARTAD value joint by assessing the ratio of average pixel intensity of the ROI to the average pixel intensity of the RR;
g. for each joint, comparing the MARTAD of the joint to a normal MARTAD value for a corresponding joint, wherein the normal MARTAD value is derived from averaging the MARTAD values for the corresponding joint from a plurality of healthy subjects: and wherein macrophage involvement is indicated by a joint specific MARTAD value that exceeds the normal MARTAD value by a predetermined threshold;
h. determining a global MARTAD value for the subject by determining the sum of difference of each of the joints of the subject that exceeds the predetermined threshold and the corresponding joint normal MARTAD value;
i. administering a course of treatment to the subject; and
j. repeating steps a) through h) and evaluating change in the global MARTAD value of the subject, wherein a decrease in global MARTAD value is indicative of efficacy of the course of treatment.

19. The method of claim 18, wherein for each joint, the joint specific RR is located within 3 ROI diameters of the ROI.

20. The method of claim 18, the mannosylated dextran construct is Tc99m-tilmanocept.
